(19) United States
(12) Patent Application Publication
(10) Pub. No.: US 2010/0168554 A1
(43) Pub. Date: Jul. 1, 2010

(54) Evaluating Cardiac Function With Dynamic Imaging Techniques and Contrast Media

(76) Inventor: Jens Sorensen, Uppsala (SE)

Correspondence Address: GE HEALTHCARE, INC., IP DEPARTMENT 101 CARNEGIE CENTER, PRINCETON, NJ, 08540-6231, US

(21) Appl. No.: 11/993,091

(22) PCT Filed: Jun. 30, 2006
(86) PCT No.: PCT/IB06/01819

§ 371 Date: Dec. 19, 2007

Related U.S. Application Data
(60) Provisional application No. 60/695,462, filed on Jun. 30, 2005.

(51) Int. Cl. A61B 5/055; A61K 49/00; A61K 51/00; A61B 6/00

(52) U.S. Cl. 600/420; 424/9.1; 424/9.3; 424/1.11; 424/1.61; 600/431

(57) ABSTRACT

Methods suitable for use in diagnostic imaging or to generate a central circulatory turnover (CCT) index for an evaluation of cardiac function of a patient are provided. The resultant CCT index will allow the clinician to integrate information reflecting the diastolic function of the heart. A computer software and kit for the evaluation of cardiac function in a patient as well as the use of a CCT index for an evaluation of cardiac function of a patient are also provided.

### FIELD OF THE INVENTION

The present invention relates to an enhancement of the non-invasive diagnosis of heart failure. It further relates to methods for the diagnostic use of dynamic imaging techniques and contrast media. More specifically, the invention relates to a method for generating a novel central circulatory turnover (CCT) index for easy and highly automated evaluation of cardiac function, using a dynamic imaging modality in combination with a contrast media such as intravenously injected indicators. Positron Emission Tomography (PET) and PET tracers are especially useful in such methods.

### BACKGROUND OF THE INVENTION

Heart failure is defined as the inability of the heart to pump sufficient amounts of blood to tissues or failure to do so without an elevation of cardiac filling pressures (Brauwald, E., ed. Heart Disease, A Textbook of Cardiovascular Medicine, 5^{th} ed. Philadelphia: WB Saunders Company, 1997). The prevalence of heart failure in the western world is 2-3% and up to 10% in the elderly. The majority of cases occur in patients with ischemic heart disease or hypertension, but the heart may also weaken when the cardiac valves are dysfunctional, in myocardial inflammation or infection and toxic degeneration of the cardiac tissue.

The main symptoms of heart failure are dyspnoea (shortness of breath due to pulmonary congestion) and fatigue (due to peripheral tissue hypoxia). In early stages, symptoms occur only during exercise. Later in the process oedema (excess tissue water) is formed and profound biochemical mechanisms are activated to compensate the reduced stroke volume. Eventually the patient dies from hypoxia. The 5-year survival rate is approximately 40%, which is comparable to many malignancies (Breast cancer 30%, Colonic cancer 50%). Heart failure is the most prevalent diagnosis in hospitalizations. The cardiac tissue architecture often deteriorates irreversibly as heart failure progresses and early diagnosis is warranted. Treatment depends on the underlying pathogenesis and can include surgery (revascularization, valvular reconstruction) and medication. Many patients are prescribed 4-5 different pharmacological agents.

Heart failure diagnosis is based on history, clinical examination and physiological tests, electrocardiography, biochemical assays and imaging studies. In acute or severe chronic cases, history and clinical examination often suffice to institute adequate symptomatic treatment. However, an objective diagnosis requires the use of one or more imaging studies. The gold standard in diagnosis is invasive catheterization of both right and left cardiac chambers to directly measure cardiac filling pressures and cardiac output, which is defined as the product of amount of the blood pumped by the heart per stroke (stroke volume) and the heart rate. However, catheterization is only used in advanced cases due to the morbidity associated with the invasiveness and the costs.

There are several non-invasive imaging modalities used clinically in diagnosing heart failure. Most non-invasive imaging modalities are not capable of measuring cardiac output and filling pressures with high precision. Various indices of cardiac function are used instead. The left ventricular ejection fraction (LV-EF), which measures contractile (systolic) function, is widely used as an index of heart failure severity. LV-EF is generally evaluated by geometrical changes in cardiac size during the cardiac cycle. The volume of the left ventricle is measured at maximal filling (end-diastolic volume) and minimal filling (end-systolic volume), and LV-EF is defined as (end-diastolic volume—end-systolic volume)/end-diastolic volume. However, LV-EF is within the normal range in 25-40% of patients with clinical heart failure (Vasan, R. S., Larson, M. G., Benjamin, E. J., Evans, J. C., Reiss, C. K. and Levy, D., Congestive Heart Failure in Subjects with Normal versus Reduced Left Ventricular Ejection Fraction: Prevalence and Mortality in Population-Based Cohort, J. Am. Coll. Cardiol., 1999; 33: 1948-55). The group of patients with normal LV-EF is believed to suffer from an abnormally elevated filling pressure, causing excess lung water and decreased blood oxygen content. Various indices of left ventricular filling velocities, which measures diastolic function, are used as substitutes of filling pressures. Currently established criteria for a diagnosis of an isolated diastolic dysfunction are fulfilled in less than 50% of cases (Cahill, J. M., Horan, M., Quigley, P., Maurer, B., and McDonald, K., Doppler-echocardiographic Indices of Diastolic Function in Heart Failure Admissions with Preserved left Ventricular Systolic Function, Eur. J. Heart. Fail. 2002; 4:473-8). This also indicates that approximately 15% of all clinically obvious heart failure cases do not obtain an objective diagnosis with the current methods.

Currently used imaging modalities in heart failure diagnosis include (ordered by frequency of clinical use):
1. Chest x-ray in the acute phase to evaluate the presence of pulmonary oedema and overall cardiac size. It is often equivocal in mild or moderate cases and does not measure cardiac function parameters.
2. Standard echocardiography is used for screening in heart failure. Interpretation can be difficult and findings are often equivocal in mild cases. Reproducibility is relatively weak. The estimated standard error (S.E.E) of LV-EF is 10%. Doppler echocardiography is, in spite of low sensitivity, the current non-invasive clinical standard method in evaluation of diastolic function.
3. Gamma camera-based radionuclide angiography is regarded as the routine clinical gold standard method in LV-EF (S.E.E. 5%) and also gives some information on ventricular velocities.
4. Gamma camera-based gated perfusion scintigraphy. LV-EF (S.E.E 8-10%). There is currently no established method of measuring global diastolic function.
5. MRI is the research gold standard in LV-EF (S.E.E 3%). There is currently no established method of measuring global diastolic function.
6. Computer tomography performs well in LV-EF (S.E.E 3-5%), but there is no established method of measuring diastolic function.

Positron emission tomography (PET) imaging is not currently used in the diagnosis of heart failure, although it has been shown to enable measurement of the LV-EF with an S.E.E of <5% with certain tracers (¹⁸F-FDG, ¹³N-ammonium). Instead, PET imaging is regarded as the research and clinical gold standard in evaluation of abnormalities in cardiac perfusion and metabolism.

PET imaging is a tomographic nuclear imaging technique that uses radioactive tracer molecules that emit positrons. When a positron meets an electron, they both are annihilated and the result is a release of energy in the form of gamma rays, which are detected by the PET scanner. By employing natural substances that are used by the body as tracer molecules, PET does not only provide information about structures in the body but also information about the physiological function of the body or certain areas therein. A common tracer molecule is for instance 2-fluoro-2-deoxy-D-glucose (FDG), which is similar to naturally occurring glucose, with the addition of an ¹⁸F-atom. Gamma radiation produced from said positron-emitting fluorine is detected by the PET scanner and shows the metabolism of FDG in certain areas or tissues of the body, e.g. in the brain or the heart. The choice of a tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule.

Commonly used radionuclides in PET tracers are ¹¹C, ¹⁸F, ¹⁵O, ¹³N or ⁷⁶Br. Recently, new PET tracers were produced that are based on radiolabelled metal complexes comprising a bifunctional chelating agent and a radiometal. Bifunctional chelating agents are chelating agents that coordinate to a metal ion and are linked to a targeting vector that will bind to a target site in the patient's body. Such a targeting vector may be a peptide that binds to a certain receptor, probably associated with a certain area in the body or with a certain disease. A targeting vector may also be an oligonucleotide specific for e.g. an activated oncogene and thus aimed for tumour localization. The advantage of such complexes is that the bifunctional chelating agents may be labelled with a variety of radiometals like, for instance, ⁶⁸Ga, ²¹³Bi or ⁸⁶Y. In this way, radiolabelled complexes with special properties may be “tailored” for certain applications.

Additionally, a number of existing tracers are also useful in this context. 15O-water, ⁸²Rb-Rubidium, ¹³N-ammonium and ¹¹C-acetate measures are used to quantify perfusion. ¹⁸FDG and ¹¹C-acetate are used to quantify various aspects of metabolism. PET is regarded as the gold standard in predicting functional improvement after revascularization in patients with prior infarctions and heart failure. However, the need for another imaging modality to assess the overall cardiac function in addition to the PET scan has lead to reluctant clinical use of this modality.

Thus, current non-invasive imaging methods for diagnosing heart failure suffer from an inability of evaluating heart failure accurately. Therefore, there is a great demand in the art for non-invasive imaging methods for easy and automatable evaluation of heart failure. In particular, there is a need for using a PET scan to assess the overall cardiac function as well as to evaluate abnormalities in cardiac perfusion and metabolism. Likewise, the PET scan would further increase clinical utility of PET.

Discussion or citation of a reference herein shall not be construed as an admission that such reference is prior art to the present invention.

### SUMMARY OF THE INVENTION

In view of the needs of the prior art, the present invention provides a method suitable for use in diagnostic imaging or to generate a central circulatory turnover (CCT) index for an evaluation of cardiac function of a patient, wherein at least one contrast media passes thru the heart and lungs of a patient and;
(i) at least one imaging modality capable of tracking and quantifying the concentration of the contrast media as the contrast media passes thru the heart and lungs;
(ii) retrieving a serial image sequence and a scanning time of the contrast media as the contrast media passes thru the heart and lungs, wherein a mean pulmonary transit time (MPTT) is calculated from the scanning time;
(iii) determining a heart rate HR of the patient from the serial image sequence and MPTT; and
(iv) calculating the CCT index of the patient.

The present invention further provides, a method for calculating the CCT index of the patient without said imaging modality. Furthermore, the CCT index is equal to 1 divided by HR times MPTT.

In yet another embodiment, a central circulatory turnover (CCT) index for evaluating cardiac function is presented.

A further embodiment of the present invention encompasses a mean pulmonary transit time (MPTT) for evaluating cardiac function.

An additional embodiment, is a computer software for calculating a CCT index for an evaluation of cardiac function of a patient, wherein the software is adapted to: store CCT index data collected during a data acquisition period.

The present invention further provides for a kit for the preparation of a CCT index for an evaluation of cardiac function of a patient.

### BRIEF DESCRIPTION OF THE FIGURES

FIG. 1 shows schematic Time-Activity curves from the right ventricular (RV) and left ventricular (LV) Region of Interest. The integrated area under the RV curve contains information of the mean radioactivity concentration during the first pass. Cardiac Output is calculated from the ratio of the injected dose and the integrated area. The solid vertical lines are the curve centroids, denoting the timepoints at which half of the injected tracer dose has passed the ventricle. The distance between the solid lines indicates the mean pulmonary transit time (MPTT). Multiplication of cardiac output with MPTT yields the cardiopulmonary distribution volume of the tracer.

FIG. 2 depicts a plot of Stroke Volume Index measured with [1-¹¹C]-acetate (SVI_{AC}) and [¹⁵O]-H₂O (SVI_{WAT}) in 26 patients with ischemic cardiomyopathy. A line of regression is included.

FIG. 3 shows a plot of Stroke Volume (SVI_{AC}) versus the Cardiopulmonary Distribution Volume of acetate (CPDVI_{AC}). All measurement are normalised to body surface area. The thick solid line represents a line of regression in groups 1 and 2.

FIG. 4 shows a plot of the weight-corrected regional pulmonary first-pass uptake of [1-11C]-acetate (LSU) against regional Lung Water (rLW) in 26 patients with ischemic cardiomyopathy. A line of regression is included.

FIG. 5 depicts a relation between Central Circulatory Turnover (CCT) to parameters of Doppler-analysis of the mitral inflow pattern. IVRT: Isovolumic Relaxation Time. DT: Mitral E-wave deceleration time. E/A-ratio: Ratio of peak velocities from early and atrial waves.

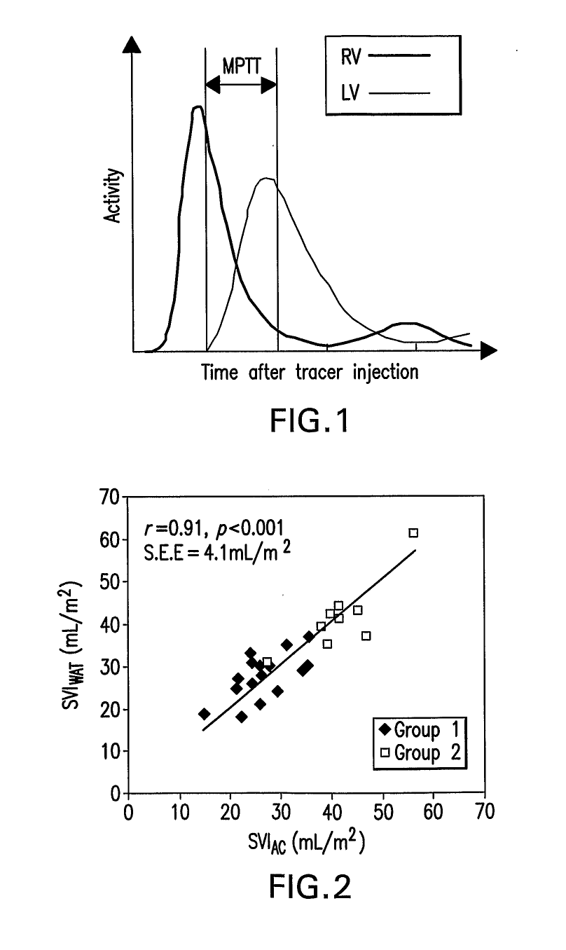

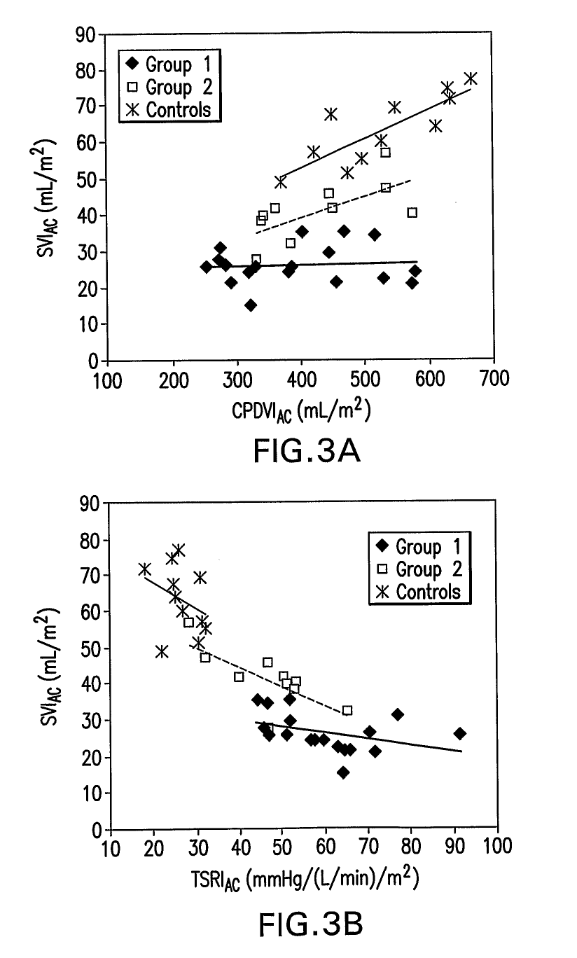

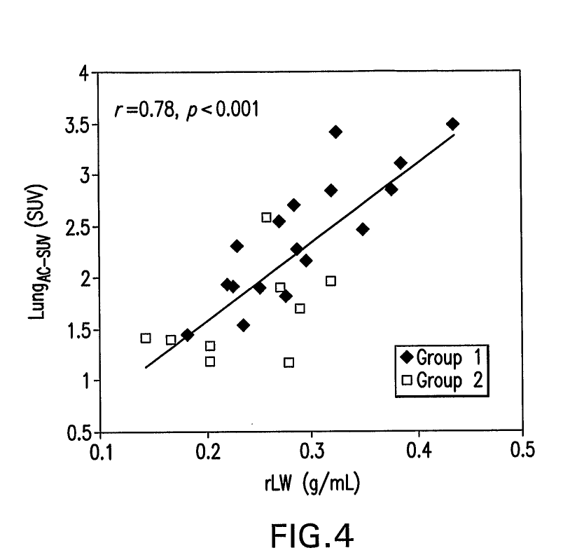

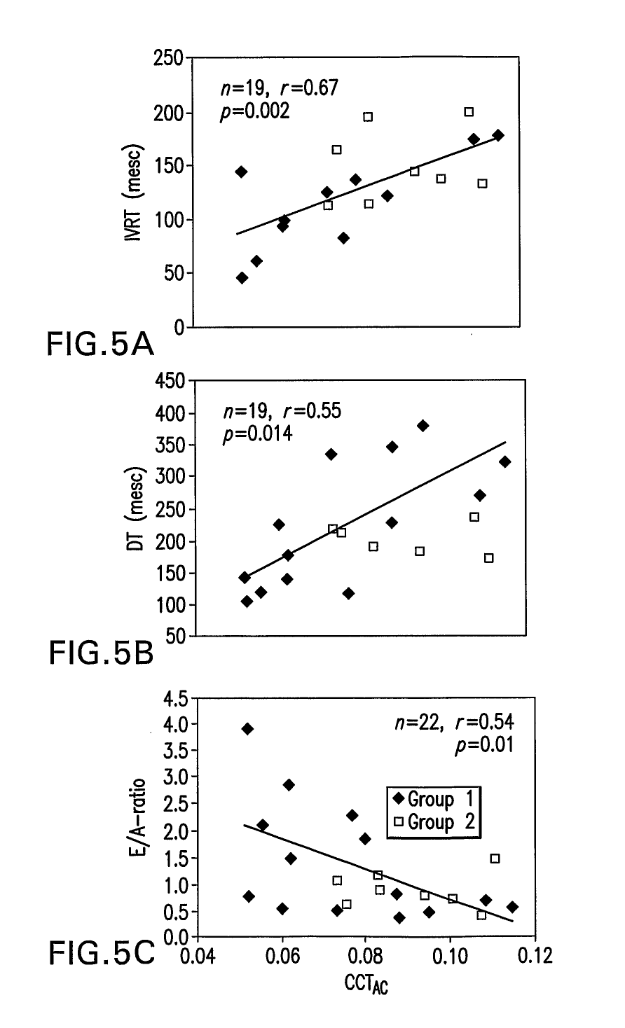

### DETAILED DESCRIPTION OF THE INVENTION

The Central Circulatory Turnover (CCT) index is a novel method for easy and highly automatable evaluation of heart failure. It is available whenever a dynamic imaging modality is used with intravenously injected indicators. Indicators in this context are defined as contrast media used in Magnetic Resonance Imaging tomography (MRI), computer tomography (CT), ultrasonography, echocardiography, or radioactive tracers used by Positron Emission Tomography (PET) and gamma-cameras.

With PET and gamma cameras, signal intensity is equal to the concentration of radioactivity (Bequerel per cc, counts per cc). With MRI, the signal intensity is related to the changes in electron spin caused by the paramagnetic properties of the contrast media (magnitude of T2-signal per cc). With computer tomography, the signal measured is the electron attenuation caused by iodinated contrast media (Hounsfield cc). With ultrasonography, the signal measured is the echogenicity of the contrast media (video-opacity per unit area).

One objective of the invention is to provide a method suitable for use in diagnostic imaging or to generate a CCT index for an evaluation of cardiac function. This objective is achieved by using an imaging modality to track and quantify the concentration of the contrast media as the contrast media passes thru the heart and lungs.

One advantage with such a method is that calculating CCT as part of a diagnostic cardiac imaging study will allow the clinician to integrate information reflecting the overall function of the heart, including the diastolic function. This will be especially useful in MRI, CT, PET, and gamma-camera-based myocardial scintigraphy (with perfusion tracers like 99m-Tc-Tetrofosmin or 211^{Th}-Thallium), where this information was not previously available. Upon the finding of an abnormally low CCT in an individual, a diagnosis of cardiac dysfunction is established.

There are several other advantages with the present method. Oncological patients often undergo several different imaging studies for diagnosis and treatment evaluations. Information of the overall cardiac function is crucial in two different scenarios: one is when the patient is selected for surgical treatment, in which case an anesthesiologist will need to know the cardiac function prior to surgery. The other possible scenario is when the patient is selected for chemotherapy.

Several chemotherapeutic agents are cardiotoxic and the cardiac function needs to be monitored thru the course of treatment. CCT, is useful in both scenarios, because this measurement can be integrated into any other study using any of the imaging modalities mentioned above. For example, serial bone scans using 99 mTc-Technetium-labeled radiopharmaceuticals are performed in almost all patients with prostaic carcinomas. When chemotherapy is introduced, the patients are also subjected to serial cardiac imaging studies to detect deteroiting cardiac function. If CCT is measured when the bone detecting agent is injected the protocol is prolonged by a few minutes, but the bone scan session will eliminate the need for the extra cardiac scan. A similar concept is possible whenever a scan including an injectable indicator is iterated for monitoring of tumor growth and there is a clinical interest in cardiac function. This possibility includes studies with gamma-camera and PET with oncologically relevant contrast media, computer tomography and MRI.

Additional advantages are achieved with the present invention wherein if CCT is measured when the bone detecting agent is injected, the protocol is prolonged by a few minutes, but the bone scan session will eliminate the need for the extra cardiac scan. A similar concept is possible whenever a scan including contrast media is iterated and there is a clinical interest in cardiac function. This possibility includes ¹⁸F-FDG-PET.

Furthermore, patients entering the emergency unit with rapid onset of dypnoea are routinely screened for the existence of pulmonary emboli. Contrast-enhanced computer tomography and MRI are the imaging modalities of choice. Both types of scans always start with a sequence of images over the heart to track the arrival of the contrast media into the pulmonary circulation. From these images, the CCT index is easily obtainable. The use of the CCT index is apparent when the patient is found not to have an embolus, because heart failure is next in line of possible conditions causing the dyspnoea. An abnormal CCT index will allow the clinician to start the correct treatment sooner at no extra costs.

Also important is the method of generating a CCT index for the evaluation of cardiac function of a patient, as exemplified by the present invention.

Below a detailed description is given of a method suitable for use in diagnostic imaging or to generate a central CCT index for an evaluation of cardiac function as described above.
In order to generate a CCT index suitable for use in diagnostic imaging in order to evaluate cardiac function of a patient, the following steps are used:
   (i) at least one contrast media, wherein the contrast media passes thru the heart and lungs of the patient;
   (ii) at least one imaging modality capable of tracking and quantifying the concentration of the contrast media as the contrast media passes thru the heart and lungs;
   (iii) retrieving a serial image sequence and a scanning time of the contrast media as the contrast media passes thru the heart and lungs, wherein a mean pulmonary transit time (MPTT) is calculated from the scanning time;
   (iv) determining a heart rate (HR) of the patient from the serial image sequence and MPTT; and
   (v) calculating the CCT index of the patient.

Yet in another embodiment, measuring MPTT of the patient is accomplished without said imaging modality. For instance, temporal changes in thoracic electrical impedance after injecting electrolytes or temporal changes in cutaneous temperature after injecting cold water are useful. Accordingly, obtaining the CCT index would not require an imaging modality. Whereby, the CCT index is equal to 1 divided by HR times MPTT.

In a further embodiment of the present invention, the imaging modality is selected from the group consisting of magnetic resonance imaging tomography, computer tomography, ultrasonography, echocardiography, and radioactive tracers used by PET and gamma cameras.

In yet another embodiment, the contrast media is an intravenously injectable indicator. Furthermore, the contrast media is selected from the group consisting of ¹⁵O-water, ⁸²Rb-Rubidium, ¹³N-ammonium, ¹¹C-acetate, ¹⁸FDG, 99 mTc-Tetrofosmin and similar radionuclides.

A further embodiment defines a MPTT as the average time taken from the contrast media to travel from point A to point B. Wherein point A is the superior vena cava, the right atrium, or the right ventricle of the heart and point B is the left atrium, the left ventricle, or the aorta of the heart.

An additional embodiment of the present invention depicts the scanning time needed to measure the MPTT is about 90 seconds. As well, the serial image sequences are obtained in about 5 seconds apart.

Additionally, in a preferred embodiment, the present invention also defines HR as the averaged time from the time of arrival of the contrast media in the right ventricle until at least 50% of the contrast media has passed from the left ventricle. The present invention further embodies the fact that HR can be achieved by counting the pulse rate manually or with a device selected from the group consisting of electrocardiography, cutaneous blood oxygen saturation pulsations, and automated sphygmomanometers.

The present invention further provides a central circulatory turnover (CCT) index for evaluating cardiac function.

The present invention also provides a mean pulmonary transit time (MPTT) for evaluating cardiac function.

In yet another embodiment, the invention provides a computer software for calculating a CCT index for an evaluation of cardiac function of a patient, wherein the software is adapted to: store CCT index data collected during a data acquisition period. The MPTT of the patient can be accomplished without said imaging modality of the patient and calculating the CCT index of the patient can be accomplished without said imaging modality.

A further embodiment of said computer software invention describes the imaging modality as being selected from the group consisting of magnetic resonance imaging tomography, computer tomography, ultrasonography, echocardiography, and radioactive tracers used by PET and gamma cameras.

Additionally, another embodiment encompasses the contrast media is an intravenously injectable indicator and is selected from the group consisting of ¹⁵O-water, ⁸²Rb-Rubidium, ¹³N-ammonium, ¹¹C-acetate, ¹⁸FDG, 99 mTc-Tetrofosmin and similar radionuclides.

Yet in another embodiment, the MPTT is the average time taken from the contrast media to travel from point A to point B.

Still in a further embodiment, point A is the superior vena cava, the right atrium, or the right ventricle of the heart and point B is the left atrium, the left ventricle, or the aorta of the heart, the scanning time needed to measure the MPTT is about 90 seconds, and the serial image sequences are obtained in about 5 seconds apart.

Yet a further embodiment of the present inventive computer software is that the HR is averaged from the time of arrival of the contrast media in the right ventricle until at least 50% of the contrast media has passed from the left ventricle, the HR is achieved by counting the pulse rate manually, and the HR is achieved with a device selected from the group consisting of electrocardiography, cutaneous blood oxygen saturation pulsations, and automated sphygmomanometers.

The present invention also provides a kit for the preparation of a CCT index for an evaluation of cardiac function of a patient wherein the MPTT of the patient is accomplished without said imaging modality of the patient.

The present inventive kit also provides for the calculation of the CCT index of the patient can be accomplished without said imaging modality and the imaging modality is selected from the group consisting of magnetic resonance imaging tomography, computer tomography, ultrasonography, echocardiography, and radioactive tracers used by PET and gamma cameras.

Yet another embodiment of the present inventive kit encompasses the contrast media as being an intravenously injectable indicator and the contrast media is selected from the group consisting of ¹⁵O-water, ⁸²Rb-Rubidium, ¹³N-ammonium, C-acetate, ¹⁸FDG, 99 mTc-Tetrofosmin and similar radionuclides.

A further embodiment said inventive kit is that the MPTT is the average time taken from the contrast media to travel from point A to point B wherein point A is the superior vena cava, the right atrium, or the right ventricle of the heart and point B is the left atrium, the left ventricle, or the aorta of the heart.

An additional embodiment encompasses the scanning time needed to measure the MPTT is about 90 seconds, the serial image sequences are obtained in about 5 seconds apart, the HR is averaged from the time of arrival of the contrast media in the right ventricle until at least 50% of the contrast media has passed from the left ventricle, the HR is achieved by counting the pulse rate manually, and the HR is achieved with a device selected from the group consisting of electrocardiography, cutaneous blood oxygen saturation pulsations, and automated sphygmomanometers.
Another embodiment of the present invention describes the use of a CCT index for in vivo diagnostics or imaging by an imaging modality such as MRI, CT, ultrasonography, echocardiography, or radioactive tracers used by PET and gamma-cameras for diseases associated with angiogenesis, cardiovascular system or other related diseases of a patient.
The method of use for generating a CCT index, wherein at least one contrast media passes thru the heart and lungs of a patient and;
   (i) at least one contrast media, wherein the contrast media passes thru the heart and lungs of the patient;
   (ii) at least one imaging modality capable of tracking and quantifying the concentration of the contrast media as the contrast media passes thru the heart and lungs;
   (iii) retrieving a serial image sequence and a scanning time of the contrast media as the contrast media passes thru the heart and lungs, wherein a mean pulmonary transit time (MPTT) is calculated from the scanning time;
   (iv) determining a heart rate (HR) of the patient from the serial image sequence and MPTT; and
   (v) calculating the CCT index of the patient for in vivo diagnostics or imaging by an imaging modality such as MRI, CT, ultrasonography, echocardiography, or radioactive tracers used by PET and gamma-cameras for diseases associated with angiogenesis and the cardiovascular system of a patient.

### EXAMPLES

The invention is further described in the following examples which is in no way intended to limit the scope of the invention.

### Example 1

### Experimental Studies

I. Central Circulatory Turnover (CCT) Studies

These studies use dynamic imaging techniques to investigate the relation of stroke volume to cardiopulmonary blood volume as an index of central circulatory dysfunction in heart failure. We have termed this relation the Central Circulatory Turnover ratio, or short: CCT. The measurement of CCT requires a stable heart rate during the first pass of tracer through the central circulation. It also requires that the mean transit time of the tracer from the right to the left ventricle is known.

CCT measurements have been performed in healthy volunteers as well as patients with heart failure of varying severity. CCT is unit less and is displayed as a fraction or percentage. In the normal condition, the CCT index is balanced at a level of 0.10 to 0.14, meaning that 10-14% of the blood volume contained in the central circulation is renewed by each heart beat. This range has been established with 11C-acetate PET in 11 elderly volunteers without a history or signs of cardiac dysfunction and in 5 young actively training endurance athletes. The athletes all had enlarged hearts and lowered left ventricular ejection fraction (LV-EF) according to echocardiographical criteria, which is a well-known false positive finding of cardiac dysfunction in highly trained individuals. However, the CCT in the athletes at rest was in the range of 0.10-0.14, not significantly different from the elderly volunteers. Furthermore, CCT was also measured during heavy supine bicycle exercises in the 5 athletes. The CCT for the athletes at rest was not significantly different from the results obtained at rest.

Early in heart failure, when the pumping capacity of the heart begins to fall, the system reacts by permanently increasing the lung blood volume to increase the filling of the left ventricle. This in turn will restore the stroke volume to some degree, due to the recoiling properties of the cardiac muscle. The CCT will change accordingly. Ten patients with mild to moderate symptoms of heart failure and slightly abnormal cardiac function, according to clinical gold standards, were evaluated with 11C-acetate PET. CCT was in the range of 0.005 to 0.12 and significantly reduced, compared to 11 elderly volunteers. In later phases of heart failure, when the stroke volume has decreased even further, the CCT will again reflect this change. Eighteen patients with symptoms and signs of severe heart failure underwent 11C-acetate PET. The CCT was in the range of 0.03 to 0.11 at rest and significantly reduced compared to both the volunteers and to the group with milder symptoms. The overall correlation coefficient of CCT versus LV-EF in these 28 patients was found to be r=0.7. The correlation coefficient of CCT versus three different standard echocardiographical indices of diastolic function was found to be r=0.5 to 0.7. LV-EF did not correlate with indices of diastolic function in this material.

Another study has been in 25 individuals, referred to myocardial scintigraphy with gamma camera and the radioactive indicator 99 mTc-Tetrofosmin. Planar images were obtained with the gamma camera every 0.25 seconds during 2 minutes after injecting the indicator. The CCT was measured at the time of the indicator injection and the LV-EF was measured with electrocardiograically-gated imaging and commercial software 30 minutes after injection. Indices of diastolic function were not available. The CCT was highly and linearly correlated with LV-EF (regression coefficient r=0.88). Also in this study, patients without symptoms or signs of heart failure had a CCT in the range of 0.10 to 0.14.

Overall, based on the material assembled so far, the CCT is highly and significantly associated with gold standard LV-EF (for both PET and gamma camera) and is also significantly associated with diastolic function (PET).

Furthermore, in the current study, it was observed by using CCT, that the cardiopulinary blood volume turnover was reduced to as little as 3% per heart beat in the patients with the most severe LV dysfunction. Since this is a factor of 4 lower than in the controls, the conclusion must be that the pulmonary blood pool appears to stagnate in heart failure even in the absence of central volume overloading. This finding is unique and could prove to be relevant to the further understanding of the pathophysiology in congestive heart failure.

As previously stated, CCT can be calculated directly from heart rate and MPTT, obviating the need for simultaneous cardiac output measurements. Hence, CCT should be obtainable with most cardiac imaging modalities that can track the passage of a tracer bolus through the heart and lungs. This also indicates that the only methodological error in CCT assessment relates to MPTT. Apart from issues related to correction for recirculation and tracer diffusibility, the time resolution of the PET scanner is the limiting factor. Based on the current results, the procedure seems adequate for hemodynamic studies by first pass analysis with PET at rest.

II. PET Imaging Analysis

A GE 4096 scanner (GE Scanditronix, Uppsala, Sweden) was used in the 28 patients. A five minute transmission scan was performed on the patient using an externally rotating ⁶⁸Ge/Ga rod. A density map thus obtained was segmented for noise reduction and used for subsequent attenuation correction of emission scans. Thirty MBq/kg of [¹⁵O]-H₂O was injected as a rapid bolus with a subsequent saline flush in an antecubital vein and the scanner was started with time frames of 20×3s, 3×10s, 4×30s and 1×120s, that were administered over 5.5 minutes to obtain a WAT-PET scan.

Fifteen minutes after the PET scan, 15 MBq/kg of [1-¹¹C]-acetate was injected in an antecubital vein as a rapid bolus with saline flush and a set of image frames with a length of 20×3s, 10×6s, 6×10s, 4×30s, 5×60s, 5×120s and 2×300s, that were adminstered over 30 minutes.

A Siemens/CTI ECAT FIR plus (CTI,/Siemens, Knoxville, Tenn.) was used in the volunteers with frame timings of 12×5s, 6×10s, 2×30s and 1×120s, that were adminstered over 5 minutes. After the initial myocardial scan in volunteers, the bed was moved to continue scanning of the abdomen and pelvis for signs of prostatic carcinoma with a routine clinical protocol.

Postprocessing of emission scans involved correction for decay, attenuation and dead time and reconstruction by filtered back projection. A Hann filter of 4.2 mm was applied and final image resolution was 8 mm in transaxial directions.

[¹¹C]-images from 2-4 minutes after injection were summed and this image was divided into short axis slices of the left ventricle. Small circular Regions of Interest (ROIs) were placed centrally in 2-5 slices of the left ventricular cavity and in the right ventricular outflow tract. A single large ROI was placed in the left lung with a margin of 2 cm towards the thoracic wall and myocardium at the level of the left atrium. All ROIs were copied to the PET scan in the patient studies. Time-activity curves (TACs) were generated from all ROIs and exported to a PC for further analysis.

III. Calculation of the CCT Index

The Mean Pulmonary Transit Time (MPTT) was calculated by the computer program by the centroid method, using linear interpolation between time-points. MPTT thereby denotes the mean time of tracer transport from the right to the left ventricle.

The Cardio-Pulmonary Distribution Volume (CPDV) was estimated as:

CPV (L)=CO (L/min)*MPTT (min)

Accordingly, a novel index called Central Circulatory Turnover Rate (CCT) was constructed as:

CCT=SV/CPDV

This index defines the fractional exchange of blood per stroke within the cardiopulmonary blood pool. CTT can be calculated by the use of HR and MPTT only:

$$\begin{matrix}C C T = \frac{SV ( mL )}{CPDV ( mL )} \\ = \frac{\frac{CO ( mL / min )}{HR ( / min )}}{CO ( mL / min ) * MPTT ( min )} \\ = \frac{1}{HR ( 1 / min ) * MRTT ( min )}\end{matrix}$$

IV. Results

Calculating CCT as part of a diagnostic cardiac imaging study will allow the clinician to integrate information reflecting the overall function of the heart, including the diastolic function. This will be especially useful in MRI, computer tomography, PET, and gamma camer-based myocardial scintigraphy (with perfusion contrast media like 99m-Tc-MyoView or 211^{Th}-Thallium), where this information was not previously available. Upon finding an abnormally low CCT in an individual, a diagnosis of cardiac dysfunction is established.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method of generating a central circulatory turnover (CCT) index for an evaluation of cardiac function of a patient, comprising the steps of:
(v) at least one contrast media, wherein the contrast media passes thru the heart and lungs of the patient;
(vi) at least one imaging modality capable of tracking and quantifying the concentration of the contrast media as the contrast media passes thru the heart and lungs;
(vii) retrieving a serial image sequence and a scanning time of the contrast media as the contrast media passes thru the heart and lungs, wherein a mean pulmonary transit time (MPTT) is calculated from the scanning time;
(viii) determining a heart rate (HR) of the patient from the serial image sequence and MPTT; and
(v) calculating the CCT index of the patient.

2. A method according to claim 1, wherein measuring the MPTT of the patient is accomplished without said imaging modality of the patient.

3. A method according to claim 2, wherein calculating the CCT index of the patient is accomplished without said imaging modality.

4. A method according to claim 1, wherein the imaging modality is selected from the group consisting of magnetic resonance imaging tomography, computer tomography, ultrasonography, echocardiography, and radioactive tracers used by PET and gamma cameras.

5. A method according to claim 1, wherein the contrast media is an intravenously injectable indicator.

6. A method according to claim 1, wherein the contrast media is selected from the group consisting of ¹⁵O-water, ⁸²Rb-Rubidium, ¹³N-ammonium, ¹¹C-acetate, ¹⁸FDG, 99 mTc-Tetrofosmin and similar radionuclides.

7. A method according to claim 1, wherein the MPTT is the average time taken from the contrast media to travel from point A to point B.

8. A method accordingly to claim 6, wherein point A is the superior vena cava, the right atrium, or the right ventricle of the heart and point B is the left atrium, the left ventricle, or the aorta of the heart.

9. A method according to claim 1, wherein the scanning time needed to measure the MPTT is about 90 seconds.

10. A method according to claim 1, wherein the serial image sequences are obtained in about 5 seconds apart.

11. A method according to claim 1, wherein the HR is averaged from the time of arrival of the contrast media in the right ventricle until at least 50% of the contrast media has passed from the left ventricle.

12. A method according to claim 1, wherein the HR is achieved by counting the pulse rate manually.

13. A method according to claim 1, wherein the HR is achieved with a device selected from the group consisting of electrocardiography, cutaneous blood oxygen saturation pulsations, and automated sphygmomanometers.

14. A central circulatory turnover (CCT) index for evaluating cardiac function according to a method of claim 1.

15. A mean pulmonary transit time (MPTT) for evaluating cardiac function according to claim 1.

16. A computer software for calculating a CCT index for an evaluation of cardiac function of a patient, wherein the software is adapted to: store CCT index data collected during a data acquisition period, wherein said CCT index comprises the steps according to claim 1.

17. The computer software for calculating a CCT index according to claim 16, wherein measuring the MPTT of the patient is accomplished without said imaging modality of the patient.

18. The computer software for calculating a CCT index according to claim 16, wherein calculating the CCT index of the patient is accomplished without said imaging modality.

19. The computer software for calculating a CCT index according to claim 16, wherein the imaging modality is selected from the group consisting of magnetic resonance imaging tomography, computer tomography, ultrasonography, echocardiography, and radioactive tracers used by PET and gamma cameras.

20. The computer software for calculating a CCT index according to claim 16, wherein the contrast media is an intravenously injectable indicator.

21. The computer software for calculating a CCT index according to claim 16, wherein the contrast media is selected from the group consisting of ¹⁵O-water, ⁸²Rb-Rubidium, ¹³N-ammonium, ¹⁸FDG, 99 mTc-Tetrofosmin and similar radionuclides.

22. The computer software for calculating a CCT index according to claim 16, wherein the MPTT is the average time taken from the contrast media to travel from point A to point B.

23. The computer software for calculating a CCT index according to claim 16, wherein point A is the superior vena cava, the right atrium, or the right ventricle of the heart and point B is the left atrium, the left ventricle, or the aorta of the heart.

24. The computer software for calculating a CCT index according to claim 16, wherein the scanning time needed to measure the MPTT is about 90 seconds.

25. The computer software for calculating a CCT index according to claim 16, wherein the serial image sequences are obtained in about 5 seconds apart.

26. The computer software for calculating a CCT index according to claim 16, wherein the HR is averaged from the time of arrival of the contrast media in the right ventricle until at least 50% of the contrast media has passed from the left ventricle.

27. The computer software for calculating a CCT index according to claim 16, wherein the HR is achieved by counting the pulse rate manually.

28. The computer software for calculating a CCT index according to claim 16, wherein the HR is achieved with a device selected from the group consisting of electrocardiography, cutaneous blood oxygen saturation pulsations, and automated sphygmomanometers.

29. A kit for the preparation of a CCT index for an evaluation of cardiac function of a patient according to the steps of claim 1.

30. The kit according to claim 29, wherein measuring the MPTT of the patient is accomplished without said imaging modality of the patient.

31. The kit according to claim 29, wherein calculating the CCT index of the patient is accomplished without said imaging modality.

32. The kit according to claim 29, wherein the imaging modality is selected from the group consisting of magnetic resonance imaging tomography, computer tomography, ultrasonography, echocardiography, and radioactive tracers used by PET and gamma cameras.

33. The kit according to claim 29, wherein the contrast media is an intravenously injectable indicator.

34. The kit according to claim 29, wherein the contrast media is selected from the group consisting of ¹⁵O-water, ⁸²Rb-Rubidium, ¹³N-ammonium, ¹¹C-acetate, ¹⁸FDG, 99 mTc-Tetrofosmin and similar radionuclides.

35. The kit according to claim 29, wherein the MPTT is the average time taken from the contrast media to travel from point A to point B.

36. The kit according to claim 29, wherein point A is the superior vena cava, the right atrium, or the right ventricle of the heart and point B is the left atrium, the left ventricle, or the aorta of the heart.

37. The kit according to claim 29, wherein the scanning time needed to measure the MPTT is about 90 seconds.

38. The kit according to claim 29, wherein the serial image sequences are obtained in about 5 seconds apart.

39. The kit according to claim 29, wherein the HR is averaged from the time of arrival of the contrast media in the right ventricle until at least 50% of the contrast media has passed from the left ventricle.

40. The kit according to claim 29, wherein the HR is achieved by counting the pulse rate manually.

41. The kit according to claim 29, wherein the HR is achieved with a device selected from the group consisting of electrocardiography, cutaneous blood oxygen saturation pulsations, and automated sphygmomanometers.

42. The use of a CCT index for in vivo diagnostics or imaging by an imaging modality such as MRI, CT, ultrasonography, echocardiography, or radioactive tracers used by PET and gamma-cameras for diseases associated with angiogenesis and the cardiovascular system of a patient.

43. The method of use for generating a CCT index according to claim 1 for in vivo diagnostics or imaging by an imaging modality such as MRI, CT, ultrasonography, echocardiography, or radioactive tracers used by PET and gamma-cameras for diseases associated with angiogenesis and the cardiovascular system of a patient.
